(19) United States
(12) Patent Application Publication
(10) Pub. No.: US 2008/0208285 A1
(43) Pub. Date: Aug. 28, 2008

(54) Methods for Treating Neurological Disorders, Including Neuropsychiatric and Neuropsychological, Disorders, and Associated Systems

(75) Inventors: Brad Fowler, Duvall, WA (US); Bradford E. Gliner, Sammamish, WA (US); W. Douglas Sheffield, Seattle, WA (US); Leif R. Sloan, Seattle, WA (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 11/961,023

(22) Filed: Dec. 20, 2007

Related U.S. Application Data
(63) Continuation of application No. 11/833,217, filed on Aug. 2, 2007.
(60) Provisional application No. 60/835,245, filed on Aug. 2, 2006.

(51) Int. Cl. A61N 1/02

(52) U.S. Cl. 607/45

(57) ABSTRACT

Methods for treating neurological disorders, including neuropsychiatric and neuropsychological disorders, and associated systems are disclosed. One such method includes identifying one or more neural populations, including a cortical target neural population, associated with a neurological condition. The method can further include comparing a patient-specific measure of a characteristic parameter for a selected one of the neural populations with a target measure for the same parameter. If the patient-specific measure differs from the target measure by at least a target amount, the method can include selecting an electrical signal polarity, frequency, or both polarity and frequency based at least in part on the difference between the patient-specific measure and the target measure. The method can further include applying electrical signals to the target neural population at the selected signal polarity, frequency, or both polarity and frequency to reduce the difference between the patient-specific measure and the target measure.

### CROSS-REFERENCE TO RELATED APPLICATION

The present application claims priority to U.S. Provisional Application No. 60/835,245, filed Aug. 2, 2006 and incorporated herein by reference.

### TECHNICAL FIELD

Aspects of the present invention are directed generally toward methods for treating neurological disorders, including neuropsychiatric and neuropsychological disorders, and associated systems.

### BACKGROUND

A wide variety of mental and physical processes are controlled or influenced by neural activity in particular regions of the brain. For example, the neural functions in some areas of the brain (i.e., the sensory or motor cortices) are organized according to physical or cognitive functions. Several areas of the brain appear to have distinct functions in most individuals. In the majority of people, for example, the areas of the occipital lobes relate to vision, the regions of the left inferior frontal lobes relate to language, and particular regions of the cerebral cortex appear to be consistently involved with conscious awareness, memory, and intellect.

Many problems or abnormalities can be caused by damage, disease and/or disorders in the brain. Disorders include neuropsychiatric and/or neuropsychological disorders, such as major depression. A person's neuropsychiatric responses may be controlled by a looped signal path between cortical structures, e.g., superficial structures at the prefrontal cortex of the brain, and deeper neural populations. For example, one such looped signal path occurs between Brodman area 9/46 at the cortex, and Brodman area 25 in the subgenual cingulate region.

Neurological problems or abnormalities are often related to electrical and/or chemical activity in the brain. Neural activity is governed by electrical impulses or “action potentials” generated in neurons and propagated along synaptically connected neurons. When a neuron is in a quiescent state, it is polarized negatively and exhibits a resting membrane potential typically between −70 and −60 mV. Through chemical connections known as synapses, any given neuron receives excitatory and inhibitory input signals or stimuli from other neurons. A neuron integrates the excitatory and inhibitory input signals it receives, and generates or fires an action potential when the integration exceeds a threshold potential. A neural firing threshold, for example, may be approximately −55 mV.

When electrical activity levels at either the superficial cortical structure or the deep brain structure are irregular, action potentials may not be generated in the normal manner. For example, action potentials may be generated too frequently, or not frequently enough. Such irregularities can result in a neuropsychiatric disorder. It follows, then, that neural activity in the brain can be influenced by electrical energy supplied from an external source, such as a waveform generator. Various neural functions can be promoted or disrupted by applying an electrical current to the cortex or other region of the brain. As a result, researchers have attempted to treat physical damage, disease and disorders in the brain using electrical or magnetic stimulation signals to control or affect brain functions.

Transcranial electrical stimulation (TES) is one such approach that involves placing an electrode on the exterior of the scalp and delivering an electrical current to the brain through the scalp and skull. Another treatment approach, transcranial magnetic stimulation (TMS), involves producing a magnetic field adjacent to the exterior of the scalp over an area of the cortex. Yet another treatment approach involves direct electrical stimulation of neural tissue using implanted deep brain stimulation electrodes (DBS). However, the foregoing techniques may not consistently produce the desired effect with the desired low impact on the patient. For example, TES may require high currents to be effective, which may cause unwanted patient sensations and/or pain. TMS may not be precise enough to target only specific areas of the brain. Deep brain stimulation is a relatively invasive procedure, and it can be difficult to accurately position DBS electrodes in tissue located well below the cortex. Accordingly, there exists a need for providing more effective, less invasive treatments for neuropsychiatric and neuropsychological disorders.

### BRIEF DESCRIPTION OF THE DRAWINGS

FIG. 1A is a schematic illustration of neurons.

FIG. 1B is a graph illustrating firing and “action potentials” associated with normal neural activity.

FIG. 2 is a schematic illustration of a system for stimulating one neural population so as to have an effect on another neural population.

FIG. 3 is a block diagram illustrating a process for affecting neural activity in accordance with an embodiment of the invention.

FIG. 4 is a flow diagram illustrating a process for applying electrical signals to cortical structures in accordance with an embodiment of the invention.

FIG. 5A is an illustration of cortical and noncortical neural pathways and neurons in an abnormal patient.

FIG. 5B-5C are schematic illustrations of the cortical and noncortical neural pathways and neurons shown in FIG. 5A under the correcting influence of electrical stimulation in accordance with particular embodiments of the invention.

FIG. 6A-6B illustrate additional or other neural populations associated with particular types of neurologic dysfunction that may be influenced or treated using electrical stimulation applied in accordance with particular embodiments of the invention, and FIG. 6C illustrates system components configured to provide and process patient information in accordance with an embodiment of the invention.

FIG. 7 illustrates an electrode device operatively coupled to an external controller in accordance with an embodiment of the invention.

FIG. 8 is a schematic illustration of a pulse system configured in accordance with several embodiments of the invention.

FIG. 9 is an isometric view of an electrode device that carries multiple electrodes in accordance with an embodiment of the invention.

### DETAILED DESCRIPTION

### Introduction

The present disclosure is directed to methods for treating neurologic dysfunction, which may include neuropsychiatric, neuropsychological, neurodevelopmental and/or other disorders; and associated systems for carrying out such methods. As used herein, the phrase “neurologic dysfunction” is used to encompass a variety of conditions or disorders, including neuropsychiatric disorders and neuropsychological disorders. As a further shorthand, the term “neuropsychiatric disorders” is used to include both neuropsychiatric disorders and neuropsychological disorders. Representative types of disorders falling within this definition include major depression, mania and other mood disorders, bipolar disorder, obsessive-compulsive disorder (OCD), Tourette's syndrome, schizophrenia, dissociative disorders, anxiety disorders, phobic disorders, post-traumatic stress disorder (PTSD), borderline personality disorder, as well as others such as Attention Deficit/Hyperactivity Disorder (ADHD) and/or craving or reward driven behaviors (e.g., associated with an addiction to legal or illegal drugs, gambling, sex, or another condition such as obesity).

In general, various aspects of the methods and systems disclosed herein are directed to treating neurological conditions or states with electrical stimulation, typically electrical stimulation applied to particular cortical structures of the patient's brain. One such method includes identifying one or more neural populations, including a first neural population, associated with the patient's condition. As discussed in greater detail below, the first neural population may be in communication with one or more other neural populations, for example, a second neural population.

In various embodiments, the first neural population includes a target neural population to which extrinsic stimulation signals may be directly or essentially directly applied. A target neural population may be identified in association with one or more neurostructural, neurofunctional, and/or neurochemical localization procedures (e.g., neural imaging procedures). Electrical signals applied to the first neural population may at least partially address the patient's condition either directly, or via an effect on the second neural population.

In general, the first neural population can include neurons or neural structures that are located within an outer, more exterior, or more superficial, or generally accessible portion of the brain, while the second neural population can include neurons or neural structures that are located within an inner, more interior, deeper, or less readily accessible portion of the brain. The first neural population can typically include neurons that are proximate or at least somewhat proximate to a region of the dura or pia mater that is exposed following a surgical burr hole or craniotomy. Moreover, the first neural population can include neurons 1) to which extrinsic stimulation signals may be directly applied using a signal delivery device (e.g., comprising a set of signal transfer devices that are at least partially carried by a generally planar support member) implanted upon or proximate to an outer surface of the brain; or 2) that can be directly affected by an electric field generated by such a signal delivery device. The first neural population can include, for example, a cortical target neural population (e.g., prefrontal cortex mediolateral front cortex, and/or orbitofrontal cortex neurons that are located within a surface-accessible gyrus) associated with a patient condition under consideration.

The second neural population can include neurons that are located in regions of the brain that are deeper or generally less directly accessible than neurons within the first neural population. The second neural population can include, for example, neurons within or generally proximate to the cingulate cortex, the hippocampus, the amygdala, the basal ganglia, the thalamus, the medial dorsal thalamus, the ventral striatum, the limbic cortex and/or other brain areas.

The method can further include comparing a patient-specific measure of a characteristic parameter for a selected one of the neural populations with a target measure for that parameter. For example, the parameter can include a relative metabolic activity level or activity level correlate of a neural population (e.g., as determined in association with a Positron Emission Tomography (PET), Single Photon Emission Computed Tomography (SPECT), functional Magnetic Resonance Imaging (fMRI), Magnetic Resonance Spectroscopy (MRS), Magnetoencephalography (MEG), electroencephalography (EEG), electrocorticography (ECoG), cerebral bloodflow (CBF) measurement, Near Infrared Optical Spectroscopy (NIRS), Optical Tomography, and/or other procedure); or a responsiveness level of a neural population. If the patient-specific measure differs from the target measure by at least a target or desired amount, the method can further include selecting an electrical signal polarity and/or frequency based at least in part on a difference, expected difference, or estimated difference between the patient-specific measure and the target measure. The method can further include applying electrical signals to the first neural population at the selected signal polarity and/or frequency to reduce the difference between the patient-specific measure and the target measure. Such electrical signals may exhibit particular stimulation parameter values or ranges intended to enhance a likelihood of achieving a desired therapeutic outcome.

### Systems and Methods for Stimulating or Affecting Particular Neural Structures

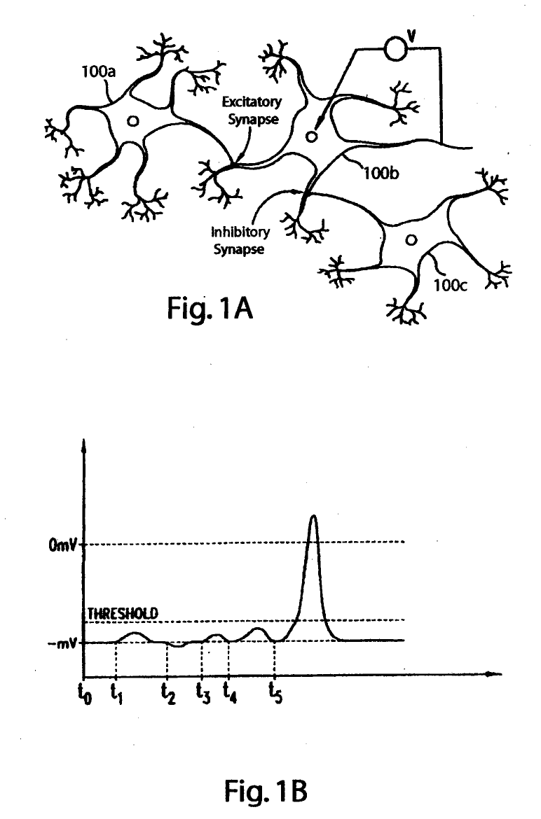

FIG. 1A is a schematic representation of several neurons 100a-100c and FIG. 1B is a graph illustrating an “action potential” related to neural activity in a normal neuron. Neural activity is governed by electrical impulses generated in neurons. For example, a first neuron 100a can send excitatory inputs to a second neuron 100b (e.g., at times t1, t3 and t4 in FIG. 1B), and a third neuron 100c can send inhibitory inputs to the second neuron 100b (e.g., at time t2 and FIG. 1B). The neurons receive and/or send excitatory and inhibitory inputs from and/or to a population of other neurons. The excitatory and inhibitory inputs influence the production of “action potentials” in the neurons, which are electrical pulses that travel through neurons by changing the flux of sodium (Na) and potassium (K) ions across the cell membrane. An action potential occurs when the resting membrane potential of the neuron surpasses a threshold level. When this threshold level is reached, an “all-or-nothing” action potential is generated. For example, as shown in FIG. 1B, the excitatory input at time t5 causes the second neuron 100b to “fire” an action potential because the input exceeds a threshold level for generating the action potential. The action potentials propagate down the length of the axon (the long portion of the neuron that makes up nerves or neuronal tracts) to cause the release of neurotransmitters from that neuron that will further influence adjacent neurons.

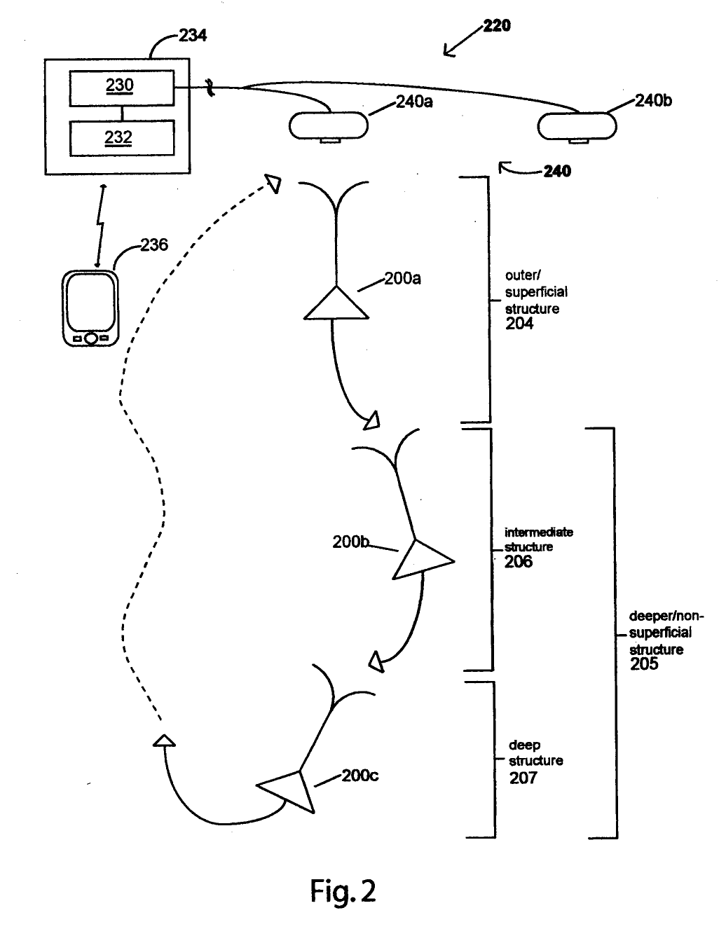

FIG. 2 is an illustration of a system 220 for modulating the activity of particular selected neurons 200a-200c in accordance with an embodiment of the invention. The individual neurons 200a-200c can form portions of larger neural populations, identified in FIG. 2 as outer or superficial structures 204 (e.g., cortical structures that are at least somewhat proximate to the dura mater directly beneath the skull) and deeper or non-superficial structures 205 (e.g., deeper cortical, subcortical, and/or deep brain structures). Superficial structures 204 may be directly affected by electrical signals from electrodes placed at appropriate epidural or subdural locations. The non-superficial structures 205 are located in more interior regions of the brain, and can include intermediate structures 206 between the superficial or outer structures 204 and deep structures 207. In a simplified representative illustration shown in FIG. 2, a superficial, generally superficial, or somewhat superficial cortical neuron 200a transmits signals to an intermediate neuron 200b, which transmits signals to a deep neuron 200c within a deep neural structure. Signals from the deep neuron 200c can be re-transmitted back to the superficial cortical neuron 200a as indicated by dashed lines in FIG. 2, optionally via other deep, intermediate and/or superficial structures.

The system 220 can include at least one signal delivery device 240 (which can include first and second signal delivery devices 240a, 240b, as shown in FIG. 2) coupled to a controller 230. The controller 230 controls the parameters in accordance with which electrical signals are issued, applied, or delivered by the signal delivery device 240. The controller 230 may be coupled to a power source 232, each of which may reside within a housing 234 that is implanted into the patient. In some embodiments, the power source 232 may be rechargeable or replenishable. Depending upon embodiment details, an electrically conductive portion of the housing 234 may serve as a remote signal transfer device or electrode for providing an electrical current return path in a unipolar stimulation configuration. The controller 230 may be configured for telemetric communication with an external programming device 236 (e.g., a computer or Personal Digital Assistant (PDA)), in a manner understood by those skilled in the relevant art.

The signal delivery device 240 can include one or more electrodes positioned to direct electrical signals to the superficial neuron 200a, which can affect the superficial neuron 200a in a manner that further affects one or more non-superficial structures 205. Accordingly, in particular embodiments, non-superficial structures 205 (e.g., deep brain structures 207) can be affected by stimulating superficial cortical structures 204 in a selected manner. This technique can be used to modulate or control a patient's neuropsychiatric and/or other condition, which may result from irregularities affecting the superficial structures 204 and/or the non-superficial structures 205.

The electrical stimulation provided to the superficial structures 204 can be provided in accordance with a wide variety of signal delivery parameters. Such parameters can include a peak current or voltage amplitude (e.g., corresponding to an initial or first pulse phase), a first phase pulse width, a pulse repetition frequency, a polarity, and/or a modulation function that may operate upon one or more parameters. However, it is believed that in at least some embodiments, the polarity of the applied signal can have a significant impact on the effect of the electrical stimulation on the superficial structures 204 (which are directly stimulated) and possibly the non-superficial structures 205 (which are affected by changes in the behavior of the superficial structures 204). The frequency of the applied signal can also have a significant impact on the effect of the electrical stimulation on the superficial and/or non-superficial structures 204, 205. Additionally, as further detailed below, the intensity or amplitude of the applied signal can significantly impact an effect of the stimulation on such structures 204, 205. Additional details regarding signal amplitude selection are included in co-pending U.S. application Ser. No. 11/773,673, filed Apr. 19, 2007 and incorporated herein by reference. The electrical stimulation may comprise charge-balanced biphasic pulses and/or other types of signals, depending upon embodiment details. The electrical stimulation may be provided at subthreshold levels and/or suprathreshold levels, with subthreshold stimulation generally having particular relevance where the signals are intended to enhance or otherwise modulate or affect neural plasticity. For example, therapeutic stimulation provided at a signal level that is approximately 25%-75% of a measured or estimated threshold signal level that by itself would be expected to activate or trigger a neural function can facilitate neuroplastic processes, particularly when the therapeutic stimulation is applied at a pulse repetition frequency of approximately 40-125 Hz, or approximately 50, 75, or 100 Hz.

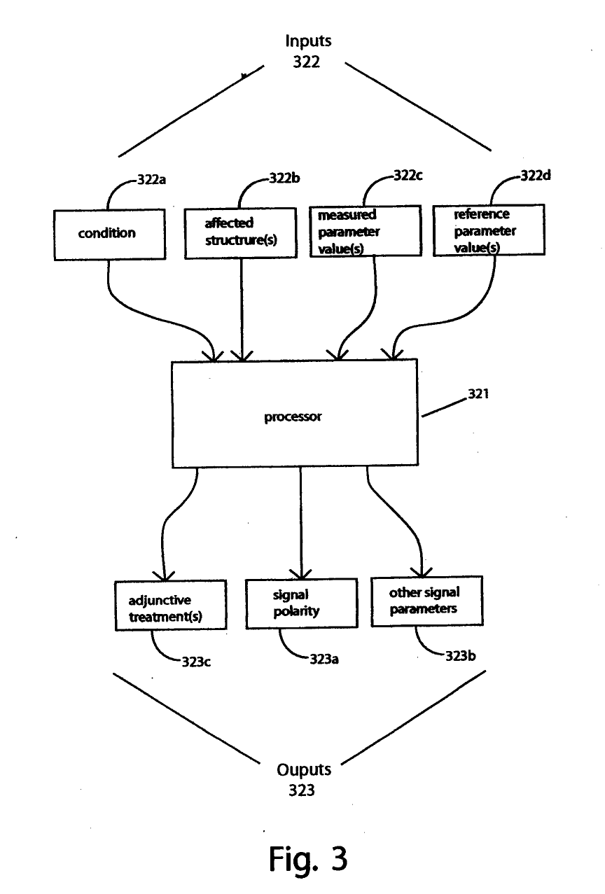

FIG. 3 is a schematic block diagram illustrating one manner in which certain treatment parameters are selected. A processor 321 (e.g., a computer processor in some embodiments, or a human processor in other embodiments) receives inputs 322 related to a particular disorder or other condition, and delivers outputs 323 corresponding to parameters for reducing or eliminating the impact of the disorder or other condition. For example, the inputs 322 can include one or more of the identity of a condition 322a from which the patient suffers, the identity and/or neural signaling characteristics of one or more affected neural structures and possibly neural pathways 322b that are adversely impacted by the condition 322a, measured (e.g., patient-specific) parameter values 322c, and reference parameter values 322d. The patient-specific parameter values 322c can include measured neural activity levels or activity level correlates, neuron responsiveness levels or responsiveness correlates, and/or other factors associated with neurological functioning. The reference parameter values 322d can include corresponding levels that are associated with the functioning of normal patients. Accordingly, for a patient suffering from a particular neurological disorder, at least some of the measured parameter values 322c will be different than the corresponding reference parameter values 322d.

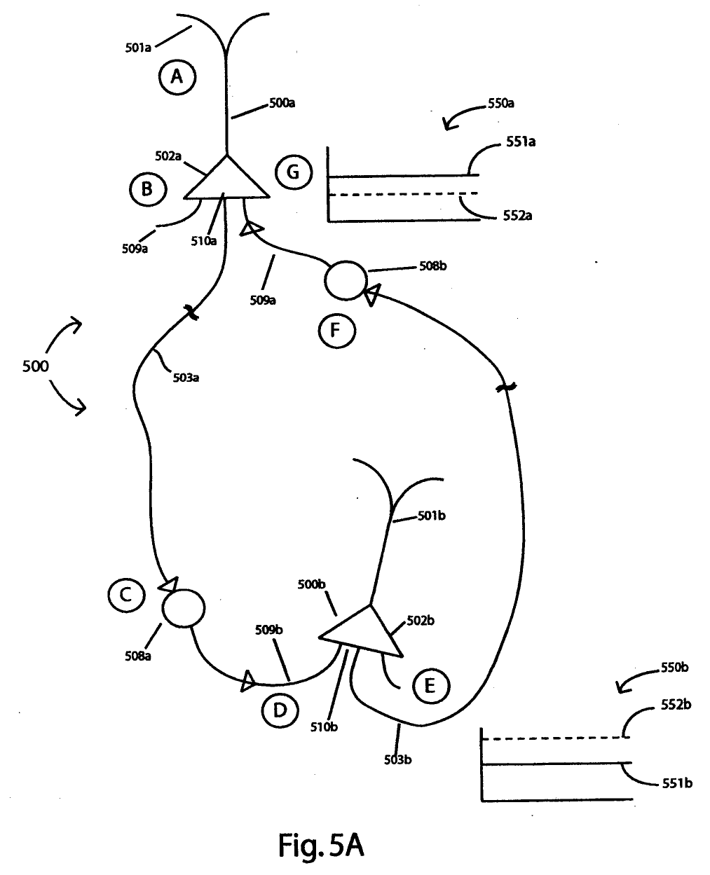

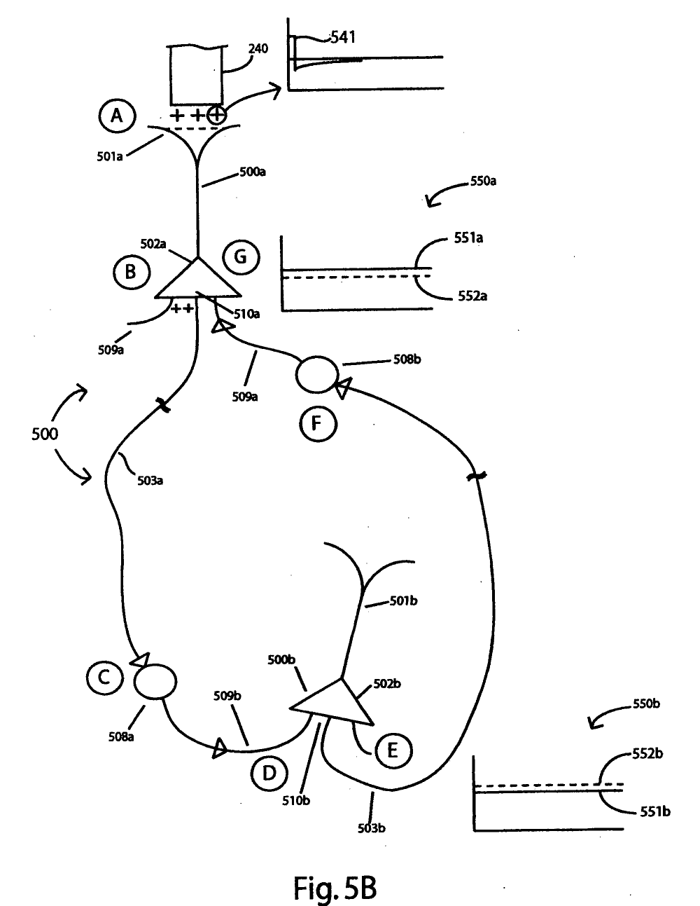

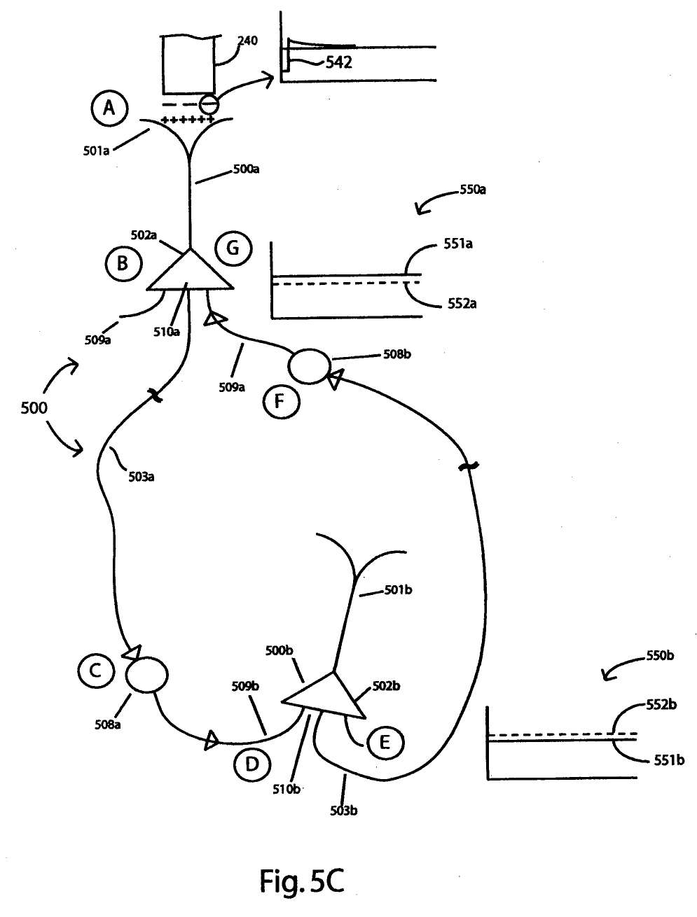

The processor 321 can receive the inputs 322 and produce the corresponding outputs 323. The outputs 323 can include a signal polarity 323a, e.g., a cathodal signal or an anodal signal. The differences between cathodal and anodal signals will be discussed in greater detail below with reference to FIGS. 5A-5C. Additional outputs 323 can include other signal parameters 323b (e.g., signal current, frequency, and voltage), and adjunctive treatments 323c. The adjunctive treatments 323c can include any type of additional treatment that may be used in conjunction or association with electrical stimulation applied in accordance with aspects of the present invention during a treatment regimen to address the patient's disorder. For example, representative adjunctive treatments include psychotherapy, cognitive behavioral therapy, counseling, medications, visualization or meditation exercises, hypnosis, memory training tasks, training tasks directed at improving the patients' ability to handle stimuli resulting in dysfunctional responses, and/or others. Additionally or alternatively, adjunctive treatment may involve one or more supplemental electromagnetic therapies such as transcranial Direct Current Stimulation (tDCS), Transcranial Magnetic Stimulation (TMS), Magnetic Seizure Therapy (MST), or electroconvulsive therapy (ECT), which typically affect neural signaling processes in a nonfocal, nonlocalized, or possibly widespread manner.

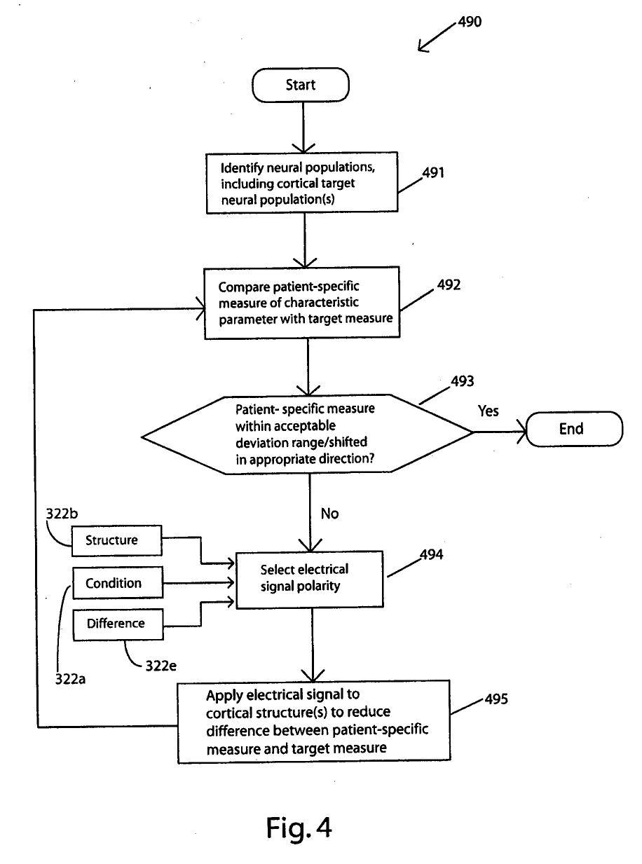

FIG. 4 is a flow diagram illustrating a representative process 490 for treating the patient in accordance with an embodiment of the invention. The process 490 can include identifying one or more neural populations, including at least one superficial cortical target neural population (process portion 491). In process portion 492, a patient-specific measure of a characteristic parameter is determined, and possibly compared with a target measure. The characteristic parameter may be associated with the target neural population, and/or a neural population that is different than the target neural population, but that may be in communication with, and affected by, the target neural population. Representative characteristic parameters include neural firing rates and/or patterns, neural metabolic activity, neural responsiveness, neuroelectric characteristics, and/or neurofunctional characteristics. If the patient-specific measure is within an acceptable deviation range from the target measure and/or has shifted appropriately (process portion 493), the process can end. Otherwise, in process portion 494, at least one of an electrical signal polarity and a signal frequency is selected. This selection can be based on the condition input 322a, the structure input 322b, and/or a difference 322e between the target measure and the patient-specific (e.g., actual) measure of the characteristic parameter. Process portion 494 can also include the selection of other signal parameters. In process portion 495, an electrical signal is applied to a superficial structure to reduce a difference between the patient-specific measure and the target measure. In general, the electrical signal inhibits or facilitates neural activity in the superficial target neural population and/or an associated non-superficial structure 205, depending upon the characteristics of the electrical signal and the characteristics of the superficial and non-superficial neural structures 204, 205. Process portions 492-495 can be repeated until the patient-specific measure of the characteristic parameter is within an acceptable deviation range of the target measure.

FIG. 5A is a simplified schematic illustration of neurons 500 and neural pathways representative of a patient suffering from a neurological disorder, for instance, depression. In one embodiment, the neurons 500 can include a superficial cortical neuron 500a (e.g., within Brodmann area 9/46) that communicates with a non-superficial neuron 500b (e.g., within Brodmann area 25). Each neuron 500a, 500b can include apical dendrites 501a, 501b, a cell body or soma 502a, 502b, an axon 503a, 503b, and one or more basal dendrites 509a, 509b. An axon hillock 510a, 510b is located proximate to the junction between the soma 502a, 502b and the corresponding axon 503a, 503b.

The neural pathway shown in FIG. 5A also includes first and second inhibitory interneurons 508a, 508b. The inhibitory interneurons 508a, 508b are located between the axon of one neuron and the basal dendrite of another. Accordingly, the inhibitory interneurons 508a, 508b receive excitatory inputs from the corresponding axon, but provide an inhibitory input to the next neuron, as is discussed further below.

Letters A-G are used in FIG. 5A and the text below to describe an expected mode of operation of the neural pathway shown in FIG. 5A in a patient experiencing neurologic dysfunction. These same reference letters are also used to describe the operation of the same neural pathway when operating under the influence of electrical signals in accordance with an embodiment of the invention, described further below with reference to FIG. 5B. Beginning with FIG. 5A, an activity level (e.g., metabolic activity level) of the superficial cortical neuron 500a may be depressed or reduced, compared to normal activity levels. This is represented by a first activity level graph 550a, in which line 551a indicates a normal metabolic activity level and line 552a indicates the actual or estimated level. Because the activity level is depressed, the axon hillock 510a (see reference letter B) tends to trigger action potentials less frequently than normal. Once action potentials are triggered at the axon hillock 510a, they proceed along the axon 503a to the first inhibitory interneuron 508a (see reference letter C). The first inhibitory interneuron 508a transmits inhibitory signals to the non-superficial neuron 500b via the corresponding basal dendrite 509b (see reference letter D).

As indicated by a second activity level graph 550b, the non-superficial neuron 500b has a heightened or hyperactive metabolic activity level 552b, which is greater than a corresponding normal level 551b. Accordingly, the non-superficial neuron 500b fires action potentials along its axon 503b on a more frequent than normal basis. Because the inhibitory signals received at its basal dendrite 509b are less frequent than normal (due to the hypoactive cortical neuron 500a), the hyperactive state of the non-superficial neuron 500b is initiated and/or maintained.

Signals triggered by the non-superficial neuron 500b are transmitted along its axon 503b (see reference letter E) to the second inhibitory interneuron 508b (see reference letter F). Because the second inhibitory interneuron 508b communicates with the basal dendrite 509a of the superficial neuron 500a (see reference letter G), the excitatory signals it receives from the non-superficial neuron 500b have an inhibitory effect on the superficial neuron 500a. This can in turn trigger, reinforce, or maintain the depressed activity level of the superficial neuron 500a described above.

FIG. 5B illustrates the same neurons and neural pathways described above with reference to FIG. 5A, with electrical stimulation provided by the signal delivery device 240, which is positioned proximate to the superficial cortical neuron 500a. It is expected that the application of an extrinsic extracellular electrical signal proximate to the apical dendrites 501a may affect voltage gated ion channels and/or result in an intracellular mobile ion gradient between the apical dendrites 501 a and the soma 502a, which may affect the neuron's internal or intrinsic signaling properties. In particular, the polarity of the applied extracellular signal can determine whether the intracellular mobile ion gradient differentially shifts membrane potentials proximate to the apical dendrites 501a and the soma 502a in a depolarizing or hyperpolarizing manner. Moreover, as further described below, additional stimulation signal parameter values or ranges (e.g., corresponding to pulse repetition frequency, peak current or voltage amplitude, or first phase pulse width) can be specified to establish, achieve, or adjust particular neural signaling properties in view of a desired therapeutic outcome.

In a particular embodiment, the signal delivery device 240 is directed to deliver anodal stimulation to the superficial neuron 500a. As used herein, the term anodal stimulation refers to stimulation having an initially positive potential. For example, as indicated graphically by an illustrative signal profile 541 in FIG. 5B, the signal delivery device 240 can deliver a series of pulses, each of which has an initial, short voltage spike with a positive polarity, followed by a longer negative polarity voltage recovery period, to provide an overall charge-balanced signal. Typically, the peak magnitude of the initial pulse phase is (significantly) greater than the peak magnitude of the recovery pulse phase. A signal transfer device that is separate, distant, or remote from the particular location at which the anodal signal is applied to the superficial cortical neuron 500a can be biased at an opposite or neutral polarity to serve as a corresponding current return path. In some embodiments, a remote signal transfer device can correspond to a portion of the housing of an implanted pulse generator. In other embodiments, the current return path can be provided one or more electrical contacts or signal transfer devices that are spaced apart (e.g., at the same, a nearby, or a distant neurofunctional region) from the signal delivery device 240 that provides the anodal stimulation.

In particular, anodal signals provided by the signal delivery device 240 proximate to the apical dendrites 501a may tend to result in an increase or accumulation of negative intracellular mobile ions within the apical dendrites 501a, which will shift the apical dendrites 501a to a more hyperpolarized state relative to their corresponding somas 502a and/or basal dendrites 509a. For example, the resting potential of the apical dendrites 501a may initially be approximately −50 to −70 mV, and the presence of the anodal signal applied to such dendrites 501 a may drive their potential more negative, e.g., toward or below −70 mV, as indicated at reference letter A. Shifting the apical dendrites 501a to a more hyperpolarized state is expected to reduce the sensitivity of such dendrites 501a to presynaptic input signals.

As indicated at reference letter B, hyperpolarizing the apical dendrites 501a is expected to induce a corresponding depolarizing shift in cellular membrane potential proximate to the soma 502a and in particular, at the axon hillock 510a, to a potential level above its normal resting value. In general, an amount of cellular membrane potential shift that will result in the generation of an action potential is lowest at or in the vicinity of the axon hillock 510a. That is, the threshold for triggering action potentials is lowest at the axon hillock 510a. The depolarizing shift proximate to the soma 502a may correspondingly raise basal dendrite membrane potentials above their normal resting values. Such a depolarizing shift may increase a likelihood of opening voltage gated ion channels within the basal dendrites 509a, thereby increasing a likelihood of generating depolarization waves within the basal dendrites 509a. In view of the foregoing, anodal stimulation applied to the apical dendrites 501a is expected to result in an increased likelihood or level of action potential generation, possibly depending upon other signal parameters, including pulse repetition frequency, which may cause the superficial cortical neuron 500a to exhibit an increased or more normal activity level. Such action potentials propagate along the corresponding axon 503a. In general, the rate of action potential generation will increase with increasing pulse repetition frequency or increasing signal intensity. One or more particular combinations of signal parameters (e.g., signal polarity, pulse repetition frequency, and amplitude) can result in an overall best, most stable, or most sustained level of therapeutic benefit, possibly in view of 1) stimulation device capabilities (e.g., power consumption) and/or 2) therapy goals. Therapy goals can include, for example, a target or desired level of dysfunction reduction as a result of ongoing (e.g., continuous or duty-cycled) stimulation; and/or a lasting therapeutic benefit (e.g., generally persisting for hours, days, weeks, months, or longer) in the absence of extrinsic neural stimulation.

In association with increased neural output from the superficial cortical neuron 500a, additional inputs may accordingly be received at the first inhibitory interneuron 508a (see reference letter C), which in turn produces an increased inhibitory effect at the soma 502b of the non-superficial neuron 500b (see reference letter D). The increased inhibitory effect reduces the cellular output or activity level 552b of the non-superficial or deep neuron 500b toward the normal level 551b. Accordingly, the non-superficial neuron 500b tends to generate fewer action potentials (reference letter E), which in turn produces a less frequent or a more normalized level of inputs to the second inhibitory interneuron 508b. The second inhibitory interneuron 508b accordingly produces a reduced or more normal level of inhibitory input to the basal dendrite 509a of the superficial cortical neuron 500a, resulting in a reduced (and therefore more normal) inhibitory effect on the superficial neuron 500a, thereby shifting the cell to a more normal activity level. This is expected to trigger and/or maintain the more normal overall activity level of the superficial neuron 500a.

One result of the stimulation protocol described above with reference to FIG. 5B is that it is expected to normalize or partially normalize the activity levels of both the superficial cortical neuron 500a and the non-superficial neuron 500b. In a particular application, the superficial neuron 500a can be located in a region corresponding to or associated with Brodmann area 9/46 of the brain (e.g., the dorsolateral prefrontal cortex (DLPFC), portions of which are associated with interpreting, evaluating, or integrating sensory system input, as well as short-term, temporary, or “working” memory), and the non-superficial neuron 500b may be located in a region corresponding to Brodmann area 25. Abnormal activity levels in both these areas, generally similar to those described above with reference to FIG. 5A, have been associated with major depression and/or other types of neurologic dysfunction. Accordingly, normalizing the activity levels in a manner identical or analogous to that described above may reduce and/or eliminate the effects of depression and/or other types of disorders.

As previously indicated, in addition to polarity, other factors can also determine or control an effect of the electrical stimulation on a target neural population, and neural populations that are in communication with the target neural population. Suitable signal parameters may include current level, voltage level, first phase pulse width, and/or pulse repetition frequency. In particular, pulse repetition frequency may be varied to achieve direct effects upon a superficial neural structure 500a, and possibly indirect effects upon other neural structures. In a particular example, at low or relatively low frequencies (e.g., between approximately 0.5 Hz to approximately 30 to 40 Hz), individual pulses may each have a “stand-alone” effect on the target neural population. That is, the effect of each pulse may be generally independent of the preceding and subsequent pulses. Depending upon the nature of a patient's neurologic dysfunction, the application of anodal signals to the apical dendrites 501a at low or very low frequencies (e.g., approximately 0.5-10 Hz) may be insufficient to raise a neural activity level by a desired amount, and may result in an overall reduction in neural activity. However, as the pulse repetition frequency increases (in the context of constant peak amplitude level and first phase pulse width), a likelihood of increasing cellular output correspondingly increases. Moreover, as the pulse repetition frequency increases, the target neural population may be subject to an overlapping or cumulative effect of the pulses. This overlapping or aggregate effect may arise as a result of overlapping intracellular depolarization waves, which may further increase a likelihood or level of action potential generation. This effect can occur at pulse frequencies of (for example) approximately 40, 50 Hz, or above or (in another example) approximately 100 Hz or above. In certain situations when pulses have a cumulative effect, the amplitude of each pulse need not be as high as it would be if each pulse were a stand-alone pulse because the combined pulses can still increase the activity level of the target neural population.

Under appropriate conditions or stimulation parameters, the application of cathodal stimulation signals to the superficial neural structures may alternatively or additionally be used to increase the activity level of a target neural population. In a manner analogous to that described above, as used herein a cathodal signal exhibits an initially negative potential. For example, as indicated graphically by an illustrative signal profile 542 in FIG. 5C, the signal delivery device 240 can deliver a series of pulses, each of which has an initial, short negative polarity voltage spike followed by a longer positive polarity voltage recovery period, to provide an overall charge-balanced signal. A signal transfer device that is separate, distant, or remote from the particular location at which a cathodal signal is applied to a superficial cortical neuron 500a may be biased at an opposite or neutral polarity to serve as a corresponding current return path.

A cathodal signal applied proximate to the apical dendrites 501a may result in an increased level of positive mobile ions within such dendrites 501a, thereby shifting the apical dendrites 501a to a more depolarized state and increasing their sensitivity to presynaptic apically-directed neural input. A corresponding intracellular mobile ion gradient may result in an increased level of negative mobile ions within or proximate to the soma 502a, which may enhance a likelihood that the soma 502a, the basal dendrites 509a, and/or the axon hillock 510a remain in a hyperpolarized state.

With an adequate, sufficient, or appropriate pulse repetition frequency, pulse amplitude, first phase pulse width, and/or signal modulation function, the depolarization state of the apical dendrites 501a can be shifted to enhance a likelihood or level of depolarization wave generation within the apical dendrites 510a. Such depolarization waves may be sufficient to trigger the generation of action potentials by the axon hillock 510a, particularly if the pulse repetition frequency ranges between approximately 40 Hz and approximately 125 Hz (e.g., 50 Hz, 75 Hz, or 100 Hz), and/or if higher pulse intensities are used than for anodal signals. In a manner analogous to that described above, a pulse repetition frequency within this range may give rise to overlapping intracellular depolarization waves of apical dendrite origin. Accordingly, the effect on the “looped” neural pathway between the superficial cortical neuron 500a and the non-superficial neuron 500b may be generally similar to, though less pronounced than, the effect described above with reference to FIG. 5B. Furthermore, cathodal signals applied at lower frequencies and/or at lower pulse intensity levels may reduce the output level and/or activity level of the target neural population (e.g., because a depolarizing shift experienced by the apical dendrites 501a can result in a hyperpolarizing shift at or near the soma 502a). Accordingly, such signals may be used in cases where the superficial cortical neuron 501a is hyperactive.

The generation of depolarization waves by the apical dendrites 501a can facilitate or enhance neural plasticity. In several embodiments, cathodal stimulation signals can be applied to the apical dendrites 501a at one or more times in association or conjunction with a set of behavioral activities (e.g., counseling or cognitive behavioral therapy) that is expected to be relevant to improving a patient's neurologic state. Cathodal stimulation may 1) enhance apical dendrite sensitivity to presynaptic input signals; and 2) increase a likelihood of generating postsynaptic depolarization waves or action potentials in response to a selective, behaviorally-driven activation of presynaptic neural pathways. This can lead to lasting, long term, or possibly permanent neuroplastic effects in the absence of extrinsic stimulation signals, where such effects may occur, for example, through Long Term Potentiation (LTP), Hebbian, or dendritically-localized Hebbian-like processes. Accordingly, the effect of behavioral therapy can be enhanced or enhanced to a greater degree by cathodal signals than by anodal signals because the apical dendrites 501a are expected to be more receptive rather than less receptive to presynaptic inputs (e.g., input signals resulting from behavioral therapy) in the presence of an extrinsic cathodal signal.

A practitioner can 1) facilitate or enhance therapeutically useful neuroplasticity or maximize a likelihood of reinforcing therapeutically beneficial neural activity; and/or 2) reduce or minimize a likelihood of reinforcing less relevant or nonbeneficial neural activity, by monitoring, estimating or measuring one or more neurofunctional, neuropsychological, or physiologic parameters through a set of behavioral and/or physiologic assessment measures during or in association with the application of extrinsic stimulation signals to the patient. Such monitoring can be particularly relevant if the patient is to receive, is receiving, or has received cathodal stimulation applied to the apical dendrites 501a. Behavioral and/or physiologic state assessment procedures can involve one or more of standard neuropsychiatric or neuropsychological tests, standard clinical assessments (e.g., the Beck Depression Inventory or Hamilton Depression Rating Scale), or structured clinical interviews; sleep monitoring or sleep architecture analysis; facial response evaluation; voice monitoring, voice signal feature analysis, or voice regulation evaluation; cardiac or pulse signal measurement; Respiratory Sinus Arrhythmia (RSA) analysis; EEG or ECoG analysis; blood oxygenation measurement; cerebral bloodflow (CBF) measurement; anatomical spectroscopy to characterize neurochemical state in particular neural regions; and/or other measures. Particular behavioral or physiologic state assessment procedures can involve short term, periodic, ongoing, or long term measurements or analyses.

In several embodiments, cathodal stimulation signals can be applied to a patient when or after a behavioral or physiologic state assessment procedure indicates that a behavioral therapy or activity acutely or historically gives rise to a therapeutic benefit for that patient. In some embodiments, cathodal stimulation signals can be applied to apical dendrites 501a in response to a medical professional's selection or specification of a stimulation mode via an external programmer 236 (e.g., at one or more times during a therapy session). In certain embodiments, cathodal stimulation signals can be applied at one or more times in an automated or semiautomated manner, possibly based upon an analysis of behavioral or physiologic state assessment procedure results (e.g., in response to the detection of particular types of temporal or spectral features or patterns within EEG or ECoG waveforms).

In the event that a behavioral or physiologic state assessment procedure indicates that a particular patient activity or emotional state is acutely or historically expected to result in a therapeutically nonbeneficial outcome, neural processes associated with or analogous to Long Term Depression (LTD) may be aided or enabled through the application of extrinsic stimulation signals to a target neural population in a pseudorandom or aperiodic manner. This can involve aperiodically varying one or more signal parameters such as pulse repetition frequency, signal polarity, signal amplitude, or signal application location relative to one or more time domains (e.g., a subseconds-based, a seconds-based, or an hours-based time domain). In a manner analogous to that described above, the application of pseudorandom or aperiodic stimulation signals to a target neural population can be based upon a medical professional's input, or an automated or semiautomated procedure responsive to behavioral or physiologic state assessment information.

In general, for a given extrinsic signal polarity and/or pulse repetition frequency, the intensity, level, or amplitude of the applied signal can affect the extent of a depolarizing or hyperpolarizing shift that particular neuronal structures experience. A higher amplitude applied signal is expected to cause a more significant cellular membrane potential shift. Depending upon embodiment details, one or more therapeutic signal levels can be determined or selected based upon a lowest or near lowest signal level at which a patient experiences a therapeutic benefit, and/or a measured or estimated threshold signal level expected to repeatably or consistently evoke or alter a given type of neural function. This neural function can relate to emotional function (e.g., mood), cognitive function (e.g., working memory or reaction time), movement, sensation, or another neural function. As representative examples, a patient might experience a mood improvement when the extrinsic signal exceeds approximately 5 mA, and a therapeutic stimulation level can accordingly be equal to or slightly greater than this level, e.g., 5.0-6.0 mA. Additionally or alternatively, the patient might experience a degradation in working memory performance, reaction time, or mood when the applied electrical signal exceeds approximately 7.0 mA, in which case the therapeutic signal level can be applied at a level below 7.0 mA (e.g., approximately 6.0 mA) for ongoing symptom management. To facilitate neuroplastic processes, a therapeutic signal having an appropriate polarity and frequency (e.g., 50-100 Hz cathodal stimulation) can be applied at approximately 20%-80% or 25%-75% (e.g., 50%) of a measured or estimated threshold signal level.

### Power Consumption and Other Considerations

Depending upon the nature of a patient's neurologic dysfunction, an extent of symptomatic reduction or improvement, patient progress over time, or other factors, a treatment program can include one or more anodal stimulation periods and/or cathodal stimulation periods. A treatment program can additionally include one or more pseudorandom or aperiodic stimulation periods. In general, anodal stimulation can be more power-efficient than cathodal stimulation as a method for increasing a likelihood or level of action potential generation, or transitioning a neural population to a more active state. Thus, in certain embodiments, anodal stimulation can be applied to the apical dendrites 501a of a target neural population outside of a patient's supervised, directed, and/or monitored behavioral activities. Cathodal stimulation can be applied during portions of one or more behavioral activities, possibly in a selectable, switchable, or programmable manner (e.g., based upon information acquired during or in association with a behavioral or physiological state assessment procedure).

Extrinsic neural stimulation can be applied to a patient in accordance with a duty cycle (e.g., continuously, or every k seconds or minutes) that provides an adequate or acceptable level of therapeutic benefit. Moreover, neural stimulation can be applied to a patient in accordance with a modulation function that establishes or modifies stimulation parameters (e.g., current or voltage level, or pulse repetition frequency) based upon a time of day, an expected chemical substance application time or metabolic half-life, or other information. In some embodiments, a neural stimulation system can include a patient based programming device (e.g., a handheld computing device coupled to a telemetry antenna) that activates a particular set of program instructions in response to patient selection of one from among a set of preprogrammed neural stimulation treatment programs. The patient based programming device may provide a graphical user interface that is responsive to user input (e.g., graphical menu selections).

In the event that a series of behavioral or physiologic state assessment procedures indicate that a patient is experiencing or attaining symptomatic benefit that persists for a period of time (e.g., minutes, hours, days, or a week or more) following an interruption of neural stimulation, a treatment program can be adjusted, modified, or appropriately duty cycled to apply stimulation signals less frequently and/or at a reduced intensity level, thereby conserving power. In certain situations, an intensity or a duty cycle corresponding to the application of (e.g., anodal) stimulation to the patient may be progressively reduced over time (e.g., several weeks, several months, or a year or longer) provided that the patient experiences longer lasting symptomatic benefit in the absence or interruption of neural stimulation over time, for example, as a result of (e.g., cathodal) stimulation applied at one or more times during regularly attended behavioral therapy sessions. In the presence of sustained symptomatic benefit, a drug or chemical substance therapy can also be modified. For example, in some cases, the patient's improvement resulting from at least some of the foregoing treatment regimens can allow the patent to reduce the intake of therapeutic drugs. In other cases, the resulting improvement can allow the patient to use therapeutic drugs that were unsuitable in the absence of the improvements, for example, if the patient was generally unresponsive to the drug prior to the improvement.

Additional/Other Neural Activity Level Considerations and/or Disorder Types

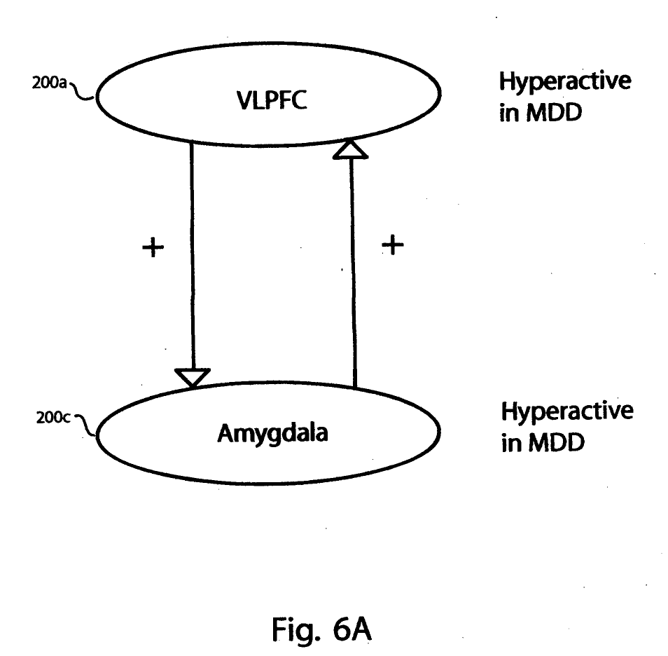

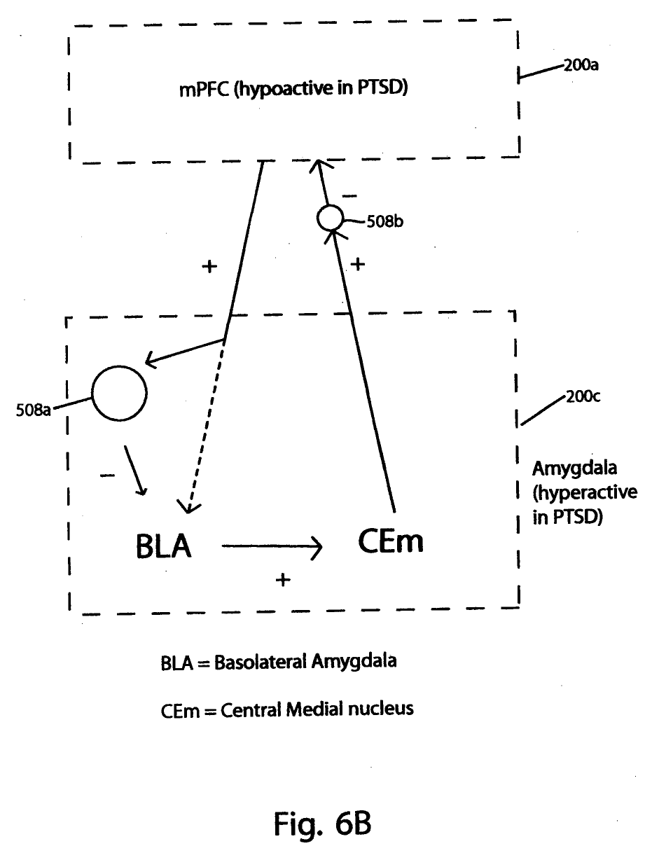

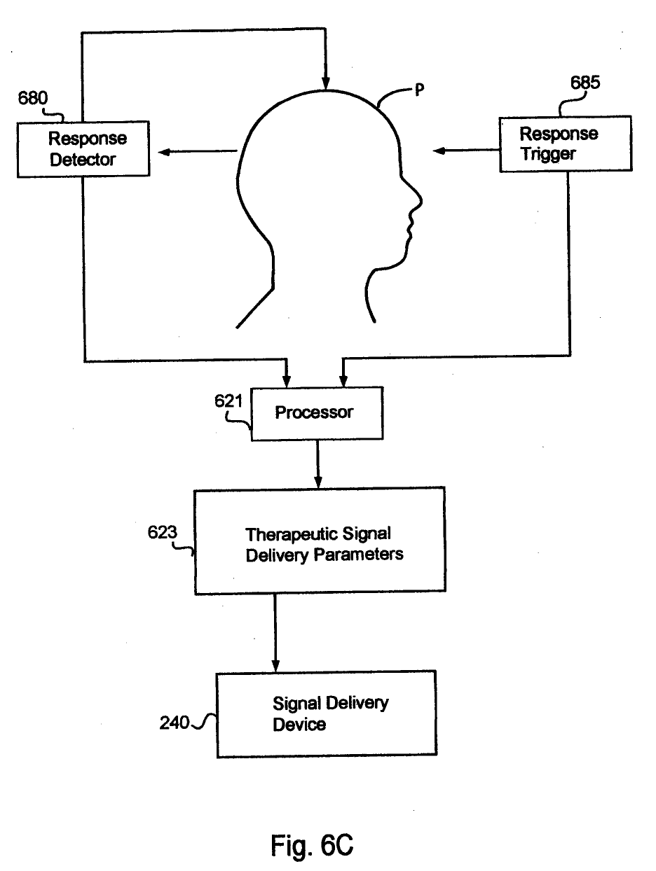

Certain types of neurologic dysfunction can additionally or alternatively be associated with superficial neural populations or structures 200a that exhibit an elevated activity level, that is, hyperactivity. For instance, as schematically illustrated in FIG. 6A, in major depressive disorder (MDD), the ventrolateral prefrontal cortex (VLPFC) may exhibit hyperactivity. Furthermore, the VLPFC maintains neural projections to the amygdala, a non-superficial neural structure 200c that may also exhibit hyperactivity associated with neurologic dysfunction arising from MDD, PTSD, or other conditions. In general, the VLPFC is associated with interpreting and planning responses to sensory system stimuli, and learning or forming new ideas, hypotheses, insights, or perceptions; and the amygdala is associated with the appraisal, generation, and maintenance of fear responses.

In order to reduce an activity level of a superficial neural structure 200a such as the VLPFC, extrinsic cathodal stimulation signals can be applied or delivered to corresponding apical dendrites. This may shift the apical dendrites to a more depolarized state, while shifting the soma to or maintaining the soma in a more hyperpolarized state. The extrinsic cathodal signals can be applied in accordance with a very low or low pulse repetition frequency (e.g., approximately 0.5-10 Hz) and possibly a low peak pulse amplitude to reduce a likelihood of generating depolarization waves within the apical dendrites that would summate and trigger action potentials. The extrinsically induced reinforcement of the soma's hyperpolarization can reduce a likelihood or level of action potential generation, which may correspondingly reduce an activity level to a more desirable or normal state.

In the event that the amygdala perceives input signals received via descending VLPFC projections (or associated intermediate structures) as excitatory or facilitatory, a decreased likelihood or level of VLPFC action potential generation may correspondingly lead to a decrease in amygdala activity, thereby shifting the amygdala to a less hyperactive or more desirable or normal state. Thus, the applied cathodal stimulation signals may indirectly reduce the amygdala's hyperactivity. In the event that the VLPFC perceives input signals received via ascending amygdala projections as excitatory or facilitatory, this reduced amygdala activity may in turn result in a (further) reduced VLPFC activity level.

As described above, the application of cathodal electrical signals to apical dendrites can facilitate or enhance neuroplasticity, particularly when associated or combined with a behavioral therapy or activity. In situations in which it may be desirable to reduce or eliminate neuroplastic effects, or when effects analogous to LTD may be desirable, the cathodal signals may be applied in a pseudorandom, aperiodic, or unpredictable manner. A controller 230 (FIG. 2) can selectively apply cathodal signals in a periodic, regular, or predicable manner or an aperiodic or unpredictable manner based upon commands received from an external programming device 236 The controller 230 can alternatively apply periodic or aperiodic signals in an automated or semiautomated manner based upon results obtained from a behavioral or physiologic state assessment procedure.

A patient can simultaneously experience dysfunctional, abnormal, or undesirable neural activity levels (e.g., as determined in association with an appropriate type of neural imaging or neuroelectric activity monitoring procedure) in two or more superficial brain regions, for example, the dorsolateral prefrontal cortex (DLPFC) and the VLPFC. In such situations, a controller 230 (FIG. 2) can direct the application of one or more types of electrical signals (e.g., anodal, cathodal, predictable/periodic, and/or unpredicatable/aperiodic) to such brain regions in a simultaneous, sequential, selectable, programmable, or other manner, possibly based upon embodiment details, the nature or severity of patient symptoms, expected or measured therapeutic benefit, power consumption, or other considerations.

As a representative example (referring back to FIG. 2), the controller 230 can enable the first signal delivery device 240a to apply anodal electrical signals to DLPFC apical dendrites outside of patient therapy sessions. The controller 230 can further enable the second signal delivery device 240b to apply aperiodic cathodal electrical signals to VLPFC apical dendrites outside of patient therapy sessions, possibly in a simultaneous or alternating manner, and/or in response to patient input received from a patient based programming device. Additionally or alternatively, during portions of a behavioral therapy session, the controller 230 can enable the first signal delivery device 240a to apply periodic cathodal electrical signals to DLPFC apical dendrites, and the second signal delivery device 240b to apply periodic or aperiodic cathodal electrical signals to VLPFC apical dendrites.

A patient having bipolar disorder can experience mood shifts or swings between depressed and euphoric states. In certain situations, depressed states can correspond to a first set of brain areas or neural populations having a first dysfunctional, abnormal, or undesirable neural activity profile, and euphoric states can correspond to a second set of neural populations having a second undesirable neural activity profile. The first and second sets of neural populations can be distinct, or have overlapping or identical cellular or neurofunctional constituencies. The controller 230 can automatically change the neural population to which electrical signals are directed, in response to a patient-initiated request, a practitioner-initiated request, and/or in response to an automatic detection of a change in patient state (e.g., via EEG/ECoG or another detection method). In still a further embodiment, the controller 230 can direct an indication to the patient that the signal delivery parameters have been changed, without actually changing the signal delivery parameters. In this case, a resulting placebo effect may still provide a therapeutic benefit to the patient.

In one embodiment, in response to patient selection of a depression treatment program via patient input received from a patient based programming device, a controller 230 can enable a first set of signal delivery devices 240a to apply electrical signals to one or more target neural populations expected to exhibit dysfunctional neural activity corresponding to depression, in a manner that beneficially alters or normalizes the dysfunctional neural activity. Similarly, in response to patient selection of a euphoria treatment program, the controller 230 can enable a second set of signal delivery devices 240b to apply electrical signals to one or more target neural populations expected to exhibit dysfunctional neural activity corresponding to euphoria, in a manner that appropriately alters or normalizes the dysfunctional neural activity. The electrical signals can be applied to superficial neural targets 200a in one or more manners identical or analogous to that described above, in accordance with an appropriate signal polarity and possibly an appropriate pulse repetition frequency value or range. For instance, if a depressed state involves a hypoactive target neural population, the electrical signals would be directed toward increasing neural activity in that target neural population. If a euphoric state involves a hyperactive target neural population, the electrical signals would be directed toward decreasing neural activity in this target neural population.

In some embodiments (for instance, an embodiment directed toward treating major depressive disorder, bipolar disorder, addiction/craving behavior, or other neurologic dysfunction), extrinsic stimulation signals can additionally or alternatively be applied to a superficial or approximately superficial target site within the orbitofrontal cortex (OFC). In general, the OFC is involved in regulating neurological reward and punishment processes. The OFC maintains dopaminergic projections to particular limbic system structures, which are associated with motivation, evaluating the emotional relevance of memories, and other functions. Neural stimulation can be applied to the OFC in one or more manners described herein to shift neural activity within the OFC and/or one or more associated non-superficial structures 205 from a dysfunctional (e.g., hyperactive or hypoactive) state toward a more normal neural activity level.

Various superficial and/or deep neural structures 200a, 200c can exhibit an abnormal level of neural activity in neurologic dysfunction associated with exposure to traumatic experience(s). FIG. 6B is a schematic illustration of a neural activity condition that can be associated with post-traumatic stress disorder (PTSD). In certain situations (e.g., traumatic event recall or processing), PTSD may involve hypoactivity in a superficial neural structure 200a known as the medial prefrontal cortex (mPFC), which in general is associated with processing the emotional content of stimuli and regulating fear responses, possibly through cognitive association processes. The mPFC may be involved in neural processes referred to as extinction, through which the emotional effects of traumatic experience may be mentally or emotionally processed or diminished. In addition to mPFC involvement, PTSD can involve hyperactivity in one or more deep or other non-superficial neural structures 200c such as the amygdala. Descending mPFC output to the amygdala primarily exerts an inhibitory or disfacilitatory effect upon the basloateral amygdala (BLA) via a first inhibitory interneuron 508a, the output of which exerts an excitatory effect upon the central medial nucleus (CEm). Ascending amygdala output from the CEm may possibly affect the mPFC in an inhibitory manner via a second inhibitory interneuron 508b.

In some embodiments, appropriate types of electrical signals (e.g., anodal or cathodal signals, as described above) can be applied to increase a likelihood or level of mPFC action potential generation, particularly when a pulse repetition frequency is above approximately 40 Hz. The increased mPFC output results in a disfacilitation of the BLA, which correspondingly reduces CEm activity. As a result of decreased CEm activity, the mPFC may experience less inhibition or disinhibition, and hence mPFC activity levels are expected to increase. Thus, electrical stimulation of the mPFC may facilitate normalization of neural activity levels in PTSD.

To facilitate or enhance neuroplasticity, cathodal stimulation signals can be applied to mPFC apical dendrites in association with or during portions of a behavioral therapy session. Additionally or alternatively, cathodal or anodal signals can be applied in an automated or semiautomated manner in response to behavioral or physiologic state assessment procedure results. Moreover, to reduce a likelihood of undesirable neuroplasticity or to aid LTD-like processes, electrical signals can be applied in an unpredictable or aperiodic manner. A controller 230 can initiate, adjust, or discontinue neural stimulation in response to patient input received via a patient based input device, for example, when a patient experiences a triggering or onset of particular emotional responses or symptoms relating to environmental stimuli or cues (e.g., certain types of unanticipated sounds). Also, neural stimulation can be applied at one or more times when a patient is at rest, likely to be asleep, or asleep in patients that experience recurring troublesome dreams, sleep disturbances, or sleep disruption in association with PTSD or other disorders.

For patients experiencing neurologic dysfunction characterized by symptoms that can be acutely triggerable (e.g., corresponding to anxiety or trauma related disorders, craving behavior, or other conditions), a set of patient-specific stimulation sites can be identified through one or more neurofunctional localization procedures. In some embodiments, a neurofunctional localization procedure can involve 1) monitoring or measuring neural parameters (e.g., neural activity or activity correlates as determined by an fMRI, PET, MEG, EEG, CBF, or other procedure; neurochemical shifts as determined by an MRS procedure; and/or an extent of neural function disruption or promotion or a shift in neuropsychiatric state following a TMS or tDCS procedure) before, during, and/or after a patient is exposed to stimuli expected to affect or evoke particular types of symptoms; and 2) identifying brain areas that seem to be involved in symptom generation or exacerbation. The stimuli can comprise sounds or images (e.g., combat recordings or footage, or images relating to substance abuse), trauma scripts (e.g., an abandonment or abuse scenario), scents, or other information or sensory system input (e.g., information that is provided to one or more sensory pathways within an individual's peripheral nervous system, and which is processed or interpreted by a brain region such as the visual cortex, the auditory cortex, the somatosensory cortex, the olfactory cortex, a given sensory association area, and/or another region) that can trigger a stress reaction, a fear response, a dissociative episode, a craving, or other response. In certain embodiments, a virtual reality device may present stimuli to the patient.

In some embodiments, a neurofunctional localization procedure can additionally identify a target site within brain region associated with processing sensory system information (e.g., a portion of the primary auditory cortex, the secondary auditory cortex, the secondary somatosensory cortex, or another brain area) that persists or remains in a “high-alert” state (e.g., a hyperactive state) for a prolonged period or long after a triggering stimulus has ceased. Extrinsic stimulation signals can be applied in one or more manners described herein (for instance, at a low pulse repetition frequency (e.g., 1-10 Hz) using an anodal polarity) to shift neurons within the target site toward a more normal level of neural activity.

Some individuals can be diagnosed with multiple types of neurologic dysfunction. For example, certain patients (e.g., “dual diagnosis” patients) can have a chemical dependency in addition to a trauma-related or other type of neuropsychiatric condition, where the chemical dependency may have developed as part of a “self medication” or other compensatory behavior. Procedures such as those described above can facilitate the identification of multiple brain areas corresponding to different (yet possibly related) dysfunctional behavior patterns or symptom profiles. A set of stimulation devices 240 can be implanted at or relative to such brain areas, and a controller 230 can facilitate signal delivery to the stimulation devices 240 at appropriate times and/or in appropriate manners. Based upon a patient's symptomatic profile, therapeutic efficacy, and/or power consumption considerations, certain of such stimulation devices 240 can apply signals to particular target neural populations on a chronic or long term basis (e.g., to address depression), while additional or other stimulation devices 240 can apply signals to target neural populations on an acute, short term, or limited duration basis (e.g., to address a triggerable anxiety condition and/or craving behavior).

FIG. 6C is a schematic illustration of system components that can be used to facilitate patient therapy in a manual, partially automated and/or automated manner. The components can include a response trigger 685, e.g., a device that provides visual, auditory, olfactory, tactile and/or other sensory stimulation to a patient P, which triggers a stress reaction, fear response, dissociative episode, craving or other response in the patient P. A response detector 680 monitors or measures the patient's response, e.g., via fMRI, PET, MEG, EEG, CBF or any of the techniques described above for identifying neural activity and/or activity correlates. A processor 621 can receive inputs from the response trigger 685 and the response detector 680. In several embodiments, the processor 621 can identify one or more stimulation sites or potential stimulation sites (e.g., by identifying areas of hypoactive and/or hyperactive neural activity). In some embodiments, the processor 621 can additionally or alternatively provide or determine an initial or an updated set of therapeutic signal delivery parameters based upon the inputs it receives from the response detector 680 and the response trigger 685. The therapeutic signal delivery parameters 623 can include electromagnetic signal polarity, amplitude, frequency, waveform type, waveform modulation function, signal duration (e.g., in accordance with a duty cycle) and/or other characteristics. The signal delivery device 240 is operatively coupled to the patient P, e.g., by being implanted in the patient P in the case of implanted electrodes, or otherwise coupled in the patient P in the case of other signal delivery modalities, including TMS or TDCS. The signal delivery device 240 can then be operated in accordance with the therapeutic signal delivery parameters 623 resulting from the patient's response to the stimulus or stimuli provided by the response trigger 685. Optionally, the foregoing components can then be used in a feedback arrangement to update the signal delivery parameters 623 and/or adjunctive therapy parameters (e.g., a drug dosage schedule), as needed, if/when the patient's responses to the response trigger 685 (or other measures of patient condition) change during the course of, or as a result of, delivering the therapeutic signals.

In view of the foregoing, in various embodiments low frequency (e.g., approximately 0.5 Hz-approximately 30 to 40 Hz, or more particularly 0.5 Hz-20 Hz or 0.5 Hz-10 Hz), anodal stimulation signals are expected to exert an inhibitory effect upon a superficial structure 204 or target neurons to which they are directly or essentially directly applied; while high frequency (e.g., above approximately 40 Hz) anodal signals can be expected to exert a facilitatory effect upon the superficial neural structure 204. In other embodiments, low frequency cathodal stimulation signals are expected to exert a somewhat inhibitory effect upon a superficial structure 204 to which the signals are applied, and high frequency cathodal stimulation signals can be expected to exert a facilitatory effect upon the superficial structure 204. High frequency cathodal signals can additionally facilitate neuroplastic processes, particularly in association or combination with behavioral activities, tasks, or therapies.

### Selection of Brain Hemisphere

Undesirable, abnormal, and/or dysfunctional neural activity can be associated with neurofunctional regions in one or both brain hemispheres. Extrinsic stimulation signals can be applied to a neural population in a particular hemisphere in one or more manners described herein to selectively inhibit or facilitate neural activity, thereby providing or reinforcing a therapeutic effect. In some situations, a given type of change in a neural function (e.g., a normalization of neural activity) resulting from the application of inhibitory or facilitatory stimulation signals to a first neural population in a first brain hemisphere can also be achieved through the application of facilitatory or inhibitory stimulation signals, respectively, to a corollary second neural population in a second brain hemisphere. For instance, one or more symptoms associated with major depressive disorder can be treated by applying facilitatory stimulation signals to portions of a patient's left DLPFC (e.g., Brodmann's area 9/46), which is generally expected to be hypoactive in most patients experiencing MDD. Some embodiments can additionally or alternatively apply inhibitory stimulation signals to portions of a patient's right DLPFC to achieve or enhance an intended therapeutic effect, possibly irrespective of whether the right DLPFC exhibits a significant degree of abnormal neural activity. Analogous considerations can apply to treating other types of neurologic dysfunction. That is, particular types of neurologic dysfunction can be treated by applying first electrical signals to a first neural population in a first hemisphere to shift neural activity in a first direction, and/or applying second electrical signals to a second neural population in a second hemisphere to shift neural activity in a second direction that is opposite to the first direction. Those of ordinary skill in the relevant art will understand that corollary brain areas in opposite hemispheres can influence or exert at least some degree of control over each other, possibly as a result of transcallosal communication and/or paradoxical facilitation phenomena.

### Representative Stimulation System Embodiments

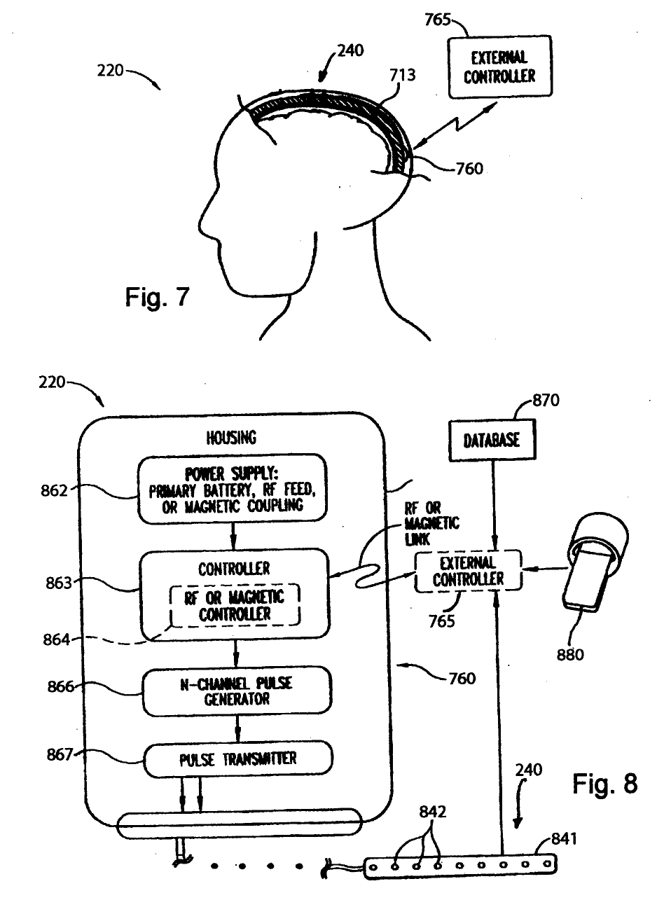

Many aspects of various techniques or procedures described above can be performed by systems similar to the system 220 introduced above with reference to FIG. 1. FIG. 7 illustrates further details of one such system. The system 220 can include a pulse system 760 that is positioned on the external surface of the patient's skull 713, beneath the scalp. In another arrangement, the pulse system 760 can be implanted at a subclavicular location. The pulse system 760 can also be controlled internally via pre-programmed instructions that allow the pulse system 760 to operate autonomously after implantation. In other embodiments, the pulse system 760 can be implanted at other locations, and at least some aspects of the pulse system 760 can be controlled externally. For example, FIG. 7 illustrates an external controller 765 that controls the pulse system 760.

FIG. 8 schematically illustrates details of an embodiment of the pulse system 760 described above. The pulse system 760 generally includes a housing 861 carrying a power supply 862, an integrated controller 863, a pulse generator 866, and a pulse transmitter 867. In certain embodiments, a portion of the housing 861 may include a signal return electrode. The power supply 862 can include a primary battery, such as a rechargeable battery, or other suitable device for storing electrical energy (e.g., a capacitor or supercapacitor). In other embodiments, the power supply 862 can include an RF transducer or a magnetic transducer that receives broadcast energy emitted from an external power source and that converts the broadcast energy into power for the electrical components of the pulse system 760.

In one embodiment, the integrated controller 863 can include a processor, a memory, and/or a programmable computer medium. The integrated controller 863, for example, can be a microcomputer, and the programmable computer medium can include software loaded into the memory of the computer, and/or hardware that performs the requisite control functions. In another embodiment identified by dashed lines in FIG. 8, the integrated controller 863 can include an integrated RF or magnetic controller 864 that communicates with the external controller 765 via an RF or magnetic link. In such an embodiment, many of the functions performed by the integrated controller 863 may be resident on the external controller 765 and the integrated portion 864 of the integrated controller 863 may include a wireless communication system.

The integrated controller 863 is operatively coupled to, and provides control signals to, the pulse generator 866, which may include a plurality of channels that send appropriate electrical pulses to the pulse transmitter 867. The pulse transmitter 867 is coupled to electrodes 842 carried by an electrode device 841. In one embodiment, each of these electrodes 842 is configured to be physically connected to a separate lead, allowing each electrode 842 to communicate with the pulse generator 866 via a dedicated channel. Accordingly, the pulse generator 866 may have multiple channels, with at least one channel associated with each of the electrodes 842 described above. Suitable components for the power supply 862, the integrated controller 863, the external controller 765, the pulse generator 866, and the pulse transmitter 867 are known to persons skilled in the art of implantable medical devices.

The pulse system 760 can be programmed and operated to adjust a wide variety of stimulation parameters, for example, which electrodes 842 are active and inactive, whether electrical stimulation is provided in a unipolar or bipolar manner, signal polarity, and/or how stimulation signals are varied. In particular embodiments, the pulse system 760 can be used to control the polarity, frequency, duty cycle, amplitude, and/or spatial and/or topographical qualities of the stimulation. Representative signal parameter ranges include a frequency range of from about 0.5 Hz to about 125 Hz, a current range of from about 0.5 mA to about 15 mA, a voltage range of from about 0.25 volts to about 10 volts, and a first pulse width range of from about 10 μsec to about 500 μsec The stimulation can be varied to match, approximate, or simulate naturally occurring burst patterns (e.g., theta-burst and/or other types of burst stimulation), and/or the stimulation can be varied in a predetermined, pseudorandom, and/or other aperiodic manner at one or more times and/or locations.

In particular embodiments, the pulse system 760 can receive information from selected sources, with the information being provided to influence the time and/or manner by which the signal delivery parameters are varied. For example, the pulse system 760 can communicate with a database 870 that includes information corresponding to reference or target parameter values. Sensors can be coupled to the patient to provide measured or actual values corresponding to one or more parameters. The measured values of the parameter can be compared with the target value of the same parameter. Accordingly, this arrangement can be used in a closed-loop fashion to control when stimulation is provided and when stimulation may cease. In one embodiment, some electrodes 842 may deliver electromagnetic signals to the patient while others are used to sense the activity level of a neural population. In other embodiments, the same electrodes 842 can alternate between sensing activity levels and delivering electrical signals. In either embodiment, information received from the signal delivery device 240 can be used to determine the effectiveness of a given set of signal parameters and, based upon this information, can be used to update the signal delivery parameters and/or halt the delivery of the signals.

In other embodiments, other techniques can be used to provide patient-specific feedback. For example, a magnetic resonance chamber 880 can provide information corresponding to the locations at which a particular type of brain activity is occurring and/or the level of functioning at these locations, and can be used to identify additional locations and/or additional parameters in accordance with which electrical signals can be provided to the patient to further increase functionality. Accordingly, the system can include a direction component configured to direct a change in an electromagnetic signal applied to the patient's brain based at least in part on an indication received from one or more sources. These sources can include a detection component (e.g., the signal delivery device and/or the magnetic resonance chamber 880).

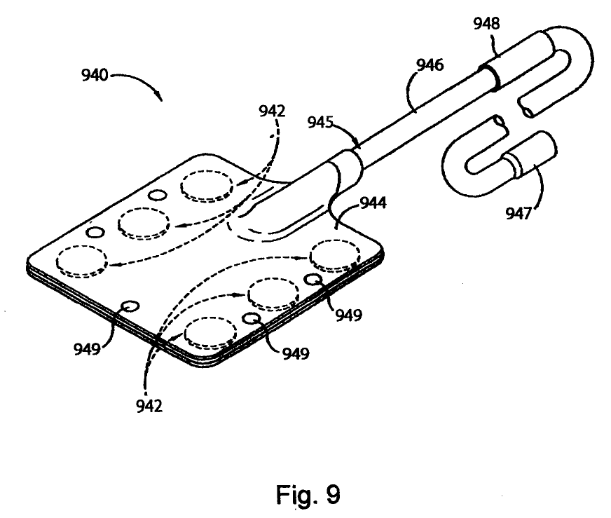

FIG. 9 is a top, partially hidden isometric view of an embodiment of a signal delivery device 940 described above, configured to carry multiple cortical electrodes 942. The electrodes 942 can be carried by a flexible support member 944 to place each electrode 942 in contact with a stimulation site of the patient when the support member 944 is implanted. Electrical signals can be transmitted to the electrodes 942 via leads carried in a communication link 945. The communication link 945 can include a cable 946 that is connected to the pulse system 760 (FIG. 8) via a connector 947, and is protected with a protective sleeve 948. Coupling apertures or holes 949 can facilitate temporary attachment of the signal delivery device 940 to the dura mater at, or at least proximate to, a stimulation site. The electrodes 942 can be biased cathodally and/or anodally. In an embodiment shown in FIG. 9, the signal delivery device 940 can include six electrodes 942 arranged in a 2×3 electrode array (i.e., two rows of three electrodes each), and in other embodiments, the signal delivery device 940 can include more or fewer electrodes 942 arranged in symmetrical or asymmetrical arrays. The particular arrangement of the electrodes 942 can be selected based on the region of the patient's brain that is to be stimulated, and/or the patient's condition.

From the foregoing, it will be appreciated that specific embodiments of the invention have been described herein for purposes of illustration, but that various modifications may be made without deviating from the invention. For example, many of the methods and systems described above may be used to treat neural populations other than those specifically described above. Aspects of the invention described in the context of particular embodiments may be combined or eliminated in other embodiments. For example, aspects of the components described with reference to FIGS. 6C-9 may be included in the system shown in FIG. 2. Further, while advantages associated with certain embodiments of the invention have been described in the context of those embodiments, other embodiments may also exhibit such advantages, and not all embodiments need necessarily exhibit such advantages to fall within the scope of the invention.

1-79. (canceled)

80. A method for treating a patient, comprising:
identifying a patient as having a neuropsychological or neuropsychiatric disorder; and
at least reducing effects of the disorder by applying electrical signals to the patient's medial prefrontal cortex.

81. The method of claim 80 wherein identifying the patient includes identifying the patient as having post-traumatic stress disorder.

82. The method of claim 80 wherein applying signals includes applying signals from a location within the patient's skull cavity and exterior to a cortical surface of the patient's brain.

83. The method of claim 80 wherein applying electrical signals includes applying electrical signals to affect a deep brain region involved in emotional functioning.

84. The method of claim 83 wherein applying electrical signals includes applying electrical signals that affect the patient's amygdala.

85. The method of claim 80 wherein applying signals includes applying signals from a location with the patient's skull and exterior to a cortical surface of the patient's brain to affect a deep brain region involved in emotional functioning.

86. The method of claim 80 wherein applying electrical signals includes increasing an activity level of the medial prefrontal cortex.

87. The method of claim 80 wherein applying electrical signals includes increasing an activity level of the medial prefrontal cortex, to in turn:
decrease an activity level of the patient's basolateral amygdala; and
increase an activity level of the patient's central medial nucleus.

88. The method of claim 80 wherein applying electrical signals includes applying anodal signals.

89. The method of claim 80 wherein applying electrical signals includes applying cathodal signals as part of a treatment regimen that also includes patient behavioral therapy.

90. The method of claim 80 wherein applying electrical signals includes applying anodal signals as part of a treatment regimen that also includes patient behavioral therapy.

91. The method of claim 80 wherein the electrical signals are first electrical signals, and wherein the method further comprises:
selecting the first electrical signals to be anodal signals and applying the first electrical signals to a cortical target neural population from a first electrical contact located within the patient's skull cavity and exterior to a cortical surface of the patient's brain to hyperpolarize dendrites of the cortical target neural population;
applying cathodal second electrical signals in addition to the anodal first electrical signals from a second electrical contact located within the patient's skull cavity and exterior to a cortical surface of the patient's brain, wherein applying the first and second electrical signal includes applying the first and second electrical signal sequentially; and
engaging the patient in an adjunctive therapy that includes at least one of psychotherapy and cognitive behavioral therapy, as part of a treatment regimen that also includes applying the second electrical signals.

92. A method for treating a patient, comprising:
identifying a patient as having an emotional functioning deficit; and
at least reducing effects of the deficit by applying electrical signals to the patient's medial prefrontal cortex to in turn affect the patient's deep brain region.

93. The method of claim 92 wherein applying signals includes applying signals from a location within the patient's skull cavity and exterior to a cortical surface of the patient's brain.

94. The method of claim 92 wherein applying electrical signals includes applying electrical signals that affect the patient's amygdala.

95. The method of claim 92 wherein applying electrical signals includes increasing an activity level of the medial prefrontal cortex.

96. The method of claim 92 wherein applying electrical signals includes increasing an activity level of the medial prefrontal cortex, to in turn:
decrease an activity level of the patient's basolateral amygdala; and increase an activity level of the patient's central medial nucleus.

97. The method of claim 92 wherein applying electrical signals includes applying anodal signals.

98. The method of claim 92 wherein the electrical signals are first electrical signals, and wherein the method further comprises:
selecting the first electrical signals to be anodal signals and applying the first electrical signals to a cortical target neural population from a first electrical contact located within the patient's skull cavity and exterior to a cortical surface of the patient's brain to hyperpolarize dendrites of the cortical target neural population;
applying cathodal second electrical signals in addition to the anodal first electrical signals from a second electrical contact located within the patient's skull cavity and exterior to a cortical surface of the patient's brain, wherein applying the first and second electrical signal includes applying the first and second electrical signal sequentially; and
engaging the patient in an adjunctive therapy that includes at least one of psychotherapy and cognitive behavioral therapy, as part of a treatment regimen that also includes applying the second electrical signals.
